(19) United States
(12) Patent Application Publication
(10) Pub. No.: US 2014/0340501 A1
(43) Pub. Date: Nov. 20, 2014

(54) SURGICAL MICROSCOPE WITH POSITIONING AID

(71) Applicant: Carl Zeiss Meditec AG, Jena (DE)

(72) Inventors: Christoph HAUGER, Aalen (DE); Christian SCHWEDES, Aalen (DE)

(73) Assignee: Carl Zeiss Meditec AG, Jena (DE)

(21) Appl. No.: 14/278,565

(22) Filed: May 15, 2014

(30) Foreign Application Priority Data
May 17, 2013 (DE) 10 2013 008 452.0

(51) Int. Cl. A61B 19/00; G02B 21/00; G03B 15/14; G02B 21/36; G02B 21/24

(52) U.S. Cl. CPC A61B 19/5223; G02B 21/36; G02B 21/241; G03B 15/14; G02B 21/0012
USPC 348/79

(57) ABSTRACT

A microscope comprises an imaging system comprising an objective having an objective lens and producing a magnified image of a focal plane of the imaging system, an image sensor disposed in the focal plane and outputting an electrical signal to the controller, and a display communicating with the controller. The controller converts signals of the image sensor into digital single images of the object, outputs the same to the display, detects a displacement of the microscope transverse to an optical axis of the objective lens relative to an object imaged, and compares the displacement with a threshold. If the displacement exceeds the threshold the controller controls a zoom system such that a total magnification is inverse monotone to a relative velocity between the microscope and the object, and/or stores the digital single images, composes them into a total image, and outputs the same to the display.

### CROSS-REFERENCES TO RELATED APPLICATIONS

The present application claims priority of Patent Application No. 10 2013 008 452.0, filed May 17, 2013 in Germany, the entire contents of which are incorporated by reference herein.

### FIELD

The present invention relates to a digital surgical microscope with a positioning aid facilitating an orientation of a user upon a change in the position and/or alignment of the surgical microscope.

### BACKGROUND

Surgical microscopes (also referred to as operating microscopes) are optical reflected-light microscopes designed for use in medical surgery and providing a magnification typically in the range from 5×-30×. Compared to other optical reflected-light microscopes, surgical microscopes use a lens system having a larger focal distance (typically a focal distance between 175 mm and 550 mm) and a respective larger working distance (distance between the objective lens of the surgical microscope located closest to an object imaged and the object). For providing a user with a three-dimensional impression of an object to be imaged, surgical microscopes are often configured as stereo(scopic) microscopes providing at least one pair of optical imaging paths for the eyes of a user, with the optical imaging paths of each pair intersecting close to a focal plane of the surgical microscope at a stereoscopic angle of between 3° and 14°. The field of view of surgical microscopes, i.e. the area located in the focal plane that can be imaged at a given time by the at least one optical imaging path onto the retina of a user, is typically larger than 1 mm². The field of view of a surgical microscope thus not only comprises a single image point as is the case with scanning microscopes; rather a multi-dimensional imaging of the object observed takes place at any point in time. Surgical microscopes are often equipped with a zoom system or a magnification changer for enabling a change in magnification, and a focusing system for changing the working distance. Regular fields of application are surgery and microsurgery. A surgical microscope is known from DE 102 008 041 284 A1 assigned to the applicant. The teaching of this document is hereby incorporated in its entirety.

In said conventional surgical microscope, the image of an object imaged with the surgical microscope is provided to a user by an eyepiece (or in stereo surgical microscopes by a pair of eyepieces). This implies that a user may not be free to move around but is required to adapt to the position and alignment of the at least one eyepiece. For facilitating an ergonomic working as well as for documentary purposes, it is known to convert the image in electrical signals with an image converter (or in stereo surgical microscopes with a stereo image converter or a pair of image converters), and to display the image to the user in addition or alternatively to the eyepieces by at least one of a monitor and a head-mounted display. A surgical microscope with a head-mounted display is known from document DE 10 2005 013 570 A1 assigned to the applicant. The teaching of this document is hereby incorporated in its entirety.

Surgical microscopes comprising image converters and no eyepieces are referred to as “digital surgical microscopes”. With digital surgical microscopes, the capturing of images using optics and image converter is spatially completely separated from an image presentation on a monitor or display.

Surgical microscopes are often supported by stands mounted to a floor or a ceiling of a treatment room or can be positioned freely across the floor of the treatment room. The stand may be adjustable manually by use of motors, and allows desired positioning and orientation of the surgical microscope above the object to be imaged. A stand is known from document DE 103 30 581 A1 assigned to the applicant. The teaching of this document is hereby incorporated in its entirety.

Despite the relative large field of view of surgical microscopes, there is sometimes the difficulty when moving the surgical microscope by use of the stand that an area of interest of an object imaged does not reside within the field of view of the surgical microscope and can not be located by a user without further ado.

### SUMMARY

Embodiments are therefore directed to a surgical microscope facilitating an orientation of a user upon a change in the position and/or alignment of the surgical microscope.

Embodiments of a surgical microscope comprise an imaging system, at least one image sensor, a controller and at least one display.

According to embodiments the imaging system comprises an objective (objective lens system) having at least one objective lens. The imaging system is configured to provide a magnified multidimensional (in particular two or three-dimensional) image of an (usually three dimensional) object disposed or disposable in a focal plane of the imaging system. According to embodiments, the focal plane of the imaging system is defined by the objective. In addition to the at least one objective lens of the objective, the imaging system may comprise one or more further optical lenses that are passed through consecutively by at least one optical imaging path. Along the at least one optical imaging path, the at least one objective lens is located closest to the object to be imaged. The optical lenses of the imaging system (including the at least one objective lens) may by simple lens elements and/or doublets (such as cemented lens elements). The imaging system may further comprise one or more optical mirror faces (such as optical mirrors and or reflecting surfaces of optical prisms, for example) consecutively folding/bending the at least one optical imaging path. According to an embodiment, the focal length of the objective comprising the at least one objective lens is of between 125 mm and 500 mm.

The at least one image sensor is disposed in a focal plane of the imaging system and outputs an electrical (and if applicable digital) signal, which enables a reconstruction—in particular one ensuring color fidelity—of the object's image generated by the imaging system. Hence, the signal output by the at least one image sensor represents the image of the object produced by the imaging system. This means that the signal output from the imaging sensor contains an information content corresponding to the information content of the image of the object generated by the imaging system to an extent enabling a reproduction of the image on a display based on the signal. The at least one image sensor may for instance be a silicon sensor, and in particular a CCD-sensor (optionally with a preceding filter wheel or color sensitive sensors instead), or an active-pixel sensor based on CMOS technology. According to an embodiment, an area of the image sensor sensitive to light has an area of at least 100×100 picture elements, and in particular of at least 320×240 picture elements.

The controller receives the signals output from the at least one image sensor and converts the same to digital single images (frames) of the object. The part of the controller dedicated to the generation of the digital single images may alternatively be formed integrally with the image sensor. The digital single images each contain the two-dimensional magnified image of the object generated by the imaging system at a respective point in time.

The at least one display is in communication with the controller, and may for instance be a monitor, a digital projector or a head-mounted display.

The controller is configured to automatically detect a displacement of the surgical microscope transverse to the optical axis of the at least one objective lens relative to the object (and thus transverse to the object to be imaged), and to compare the detected displacement with a threshold. Hereby only the component of the displacement is considered the direction of which is perpendicular to the optical axis of the at least one objective lens and is thus oriented transverse (e.g. orthogonal) to the at least one optical imaging path. The detected displacement of the surgical microscope relative to the object to be imaged may be caused by at least one of an absolute displacement of the surgical microscope and an absolute displacement of the object to be imaged.

According to an embodiment, the threshold is defined such that a displacement of the surgical microscope transverse to the optical axis of the at least one objective lens due to vibrations and oscillations of the surgical microscope does not exceed the threshold. According to an embodiment, the threshold is defined such that a displacement of the surgical microscope relative to the object to be imaged due to a displacement of the object to be imaged caused by periodic oscillations/movements of the object (for instance due to a patient's respiration or heartbeat) does not exceed the threshold. According to an embodiment, the threshold defines a minimum value for the displacement of the surgical microscope relative to the object to be imaged of in particular at least 0.25 cm, and further in particular of at least 0.5 cm, and/or the threshold defines a minimum period of time for the displacement of the surgical microscope relative to the object to be imaged of in particular at least 0.25 seconds, and further in particular of at least 0.5 seconds, and further in particular of at least 1 second. The threshold may be specified in the controller or be settable by a user via a user interface.

According to an embodiment, only the part of the displacement of the surgical microscope relative to the object to be imaged is considered which results from a displacement of the field of view in the focal plane of the surgical microscope.

According to an embodiment, the imaging system comprises at least one optical zoom (lens) system in addition to the objective with the objective enabling a change in the working distance and the zoom system enabling a change in magnification. The controller is further configured to automatically control the at least one zoom system of the imaging system in the event of a displacement transverse to the optical axis of the at least one objective lens (e.g. orthogonal to the optical axis of the at least one objective lens) exceeding the threshold such that a magnification effected by the imaging system as a whole depends inversely monotonically from a relative velocity between the surgical microscope and the object to be imaged in a direction transverse to the optical axis of the at least one objective lens. This means that the magnification provided by the imaging system as a whole is selected by the controller the lower, the higher the relative velocity between the surgical microscope and the object to be imaged is. The magnification decreases as the velocity increases and the magnification increased as the velocity decreases. Hereby only the component of the velocity is considered, that is oriented in a direction transverse to the optical axis of the at least one objective lens (e.g. orthogonal to the optical axis of the at least one objective lens) and thus transverse to the at least one optical imaging path. According to an embodiment, the controller adapts the magnification to the velocity not continuously but stepwise.

Alternatively or additionally, the controller is adapted to automatically store the digital single images in the event of a displacement transverse to the optical axis of the at least one objective lens (e.g. orthogonal to the optical axis of the at least one objective lens) exceeding the threshold in a memory that may be incorporated into the controller, and to combine the stored single images automatically to one total (overall) image of the object to be imaged. A total image correspondingly composed from single images is also referred to as a panoramic image. According to an embodiment, the single images are combined to form a total image by “image stitching”, using an image processing software running on the controller.

Finally, the controller is adapted to output the digital single images and/or the total image to the at least one display for the purpose of being presented to a user.

Since the surgical microscope described above automatically enables, in the event of a displacement of the surgical microscope, a larger overview of the object to be imaged (by selecting a lower magnification and thus providing a larger field of view and/or by providing the total image composed from several single images), a desired arrangement of the field of view of the imaging system with respect to the object to be imaged is facilitated for a user. This is particularly advantageous when using small and lightweight surgical microscopes, and in particular when the surgical microscope is moved manually.

According to an embodiment, the controller is configured to automatically detect the displacement of the surgical microscope transverse to the optical axis of the at least one objective lens by comparing temporally consecutive digital single images produced from electrical signals of the at least one image sensor. This may for instance be implemented by identifying identical image contents in single images following each other in time and by comparing their positions in the temporally consecutive single images. By considering the magnification of the imaging system respectively used when capturing the digital single images, whereby the magnification can be stored together with the digital single images, it is possible to calculate the distance by which the surgical microscope was moved in the focal plane relative to the object to be imaged from the differences in the positions of the identical image contents in the temporally consecutive single images. Together with an additional time measurement the controller may optionally also determine the speed of the displacement of the surgical microscope in the focal plane relative to the object to be imaged from the thus determined distance. According to an embodiment, the time measurement is implied by the frequency of images generated by the at least one image sensor. Accordingly, no additional sensor is required for detecting the displacement and speed relative to the object to be imaged.

According to an embodiment, the surgical microscope further comprises at least one acceleration sensor for detecting an acceleration of the surgical microscope. To this respect, the acceleration sensor may in particular be disposed nearby the imaging system. The controller is able to automatically determine a displacement of the surgical microscope transverse to the optical axis of the at least one objective lens based on the acceleration detected with the acceleration sensor. Together with an additional time measurement, the controller is optionally also able to calculate the displacement speed and the distance covered.

According to an embodiment, the controller is adapted to automatically control the at least one zoom system of the imaging system during a displacement of the surgical microscope transverse to the optical axis of the at least one objective lens relative to the object to be imaged exceeding the threshold such that the magnification provided by the imaging system as a whole is inversely proportional to a relative velocity between the surgical microscope and the object to be imaged.

According to an alternative embodiment, the controller is adapted to automatically control the at least one zoom system of the imaging system during a displacement of the surgical microscope transverse to the optical axis of the at least one objective lens relative to the object to be imaged exceeding the threshold such that the magnification provided by the imaging system as a whole is minimal during the entire displacement.

According to an embodiment, the controller is adapted to automatically control the at least one zoom system of the imaging system after a displacement of the surgical microscope transverse to the optical axis of the at least one objective lens exceeding the threshold such that the magnification of the imaging system selected at the start of the displacement is restored. After a displacement, a user may therefore immediately continue working with the magnification used before the displacement.

According to an embodiment, the controller is configured to automatically control the objective of the imaging system such that the object to be imaged continuously resides within the focal plane of imaging system and hence also during the entire period of the displacement of the surgical microscope transverse to the optical axis of the at least one objective lens. Hence, the imaging system produces a sharp image of the object to be imaged also during the displacements. A respective functionality is also referred to as autofocus.

According to an embodiment, the controller is adapted to automatically control the display during a displacement of the surgical microscope transverse to the optical axis of the at least one objective lens exceeding the threshold such that a marker (for instance crosshairs) is superimposed to a center of the image shown on the display. According to an embodiment, the marker is positioned in the image shown on the display to mark the point on the object to be imaged at which the optical axis of the objective lens intersects the object to be imaged.

According to an embodiment, the controller is configured to automatically control the display after a displacement of the surgical microscope transverse to the optical axis of the at least one objective lens exceeding the threshold such that the total image generated during the preceding displacement is shown in addition to the single images currently generated from the signals output from the at least one image sensor. This simultaneous presentation of the total image and the single images may for instance be implemented side-by-side or picture-in-picture.

According to an embodiment, the controller is configured to automatically effect an image reversal and/or rotation of the single images generated from the signals output from the at least one image sensor. Hence, since a possibly required image reversal or rotation is implemented digitally by the controller and not optically, the imaging system may accordingly be designed smaller and more lightweight.

According to an embodiment, the imaging system provides at least one pair of optical imaging paths intersecting near the focal plane of the imaging system at a stereoscopic angle of between 3° and 14°. The surgical microscope may therefore be implemented in the form of a stereoscopic surgical microscope. In this case, the surgical microscope comprises either at least one pair of image sensors with each sensor assigned to one of the optical image paths, or the at least one image sensor has a sensor area big enough for receiving both images of the object to be imaged produced by the two optical imaging paths. The at least one display may in this case be further adapted for a 3D presentation.

According to an embodiment, the controller automatically controls the at least one image sensor of the surgical microscope during a displacement transverse to the optical axis of the at least one objective lens exceeding the threshold such that, during the displacement, the image sensor continuously or quasi-continuously (i.e. in real time) outputs electrical signals to the controller that are converted by the controller in a plurality of multidimensional, time-shifted digital single pictures of the object's image produced by the image system.

According to an embodiment, the surgical microscope is supported by a stand. The stand my be attached stationary on a wall, a floor, or a ceiling, or be displaceable on rollers.

According to an embodiment, the surgical microscope is a digital surgical microscope the imaging system of which comprises no eyepieces.

It is noted that the above embodiments may be combined in any way. It is further noted that the object to be imaged does not form part of the surgical microscope claimed. The object to be imaged may be located in the focal plane or not be present at all.

The terms “including”, “comprising”, “containing”, “having” and “with”, as well as grammatical modifications thereof used in this specification and the claims for listing features, are generally to be considered to specify a non-exhaustive listing of features like for instance method steps, components, ranges, dimensions or the like, and do by no means preclude the presence or addition of one or more other features or groups of other or additional features.

### BRIEF DESCRIPTION OF THE DRAWINGS

The forgoing as well as other advantageous features of the disclosure will be more apparent from the following detailed description of exemplary embodiments together with the claims and the Figures. In the Figures, like or similar elements are indicated by like or similar reference signs. It is noted that the invention is not limited to the embodiments of the exemplary embodiments described, but is defined by the scope of the enclosed claims, and that not all possible embodiments necessarily exhibit each and every, or any, of the advantages identified herein. In particular, embodiments according to the invention may implement individual features in a different number and combination than the examples instanced below. In the following explanation of an exemplary embodiment of the invention, it is referred to the enclosed Figures, of which

FIG. 1 shows a schematic representation of an application of a surgical microscope according to an embodiment of the invention;

FIG. 2 shows a schematic representation of the structure of the surgical microscope of FIG. 1,

FIG. 3a shows a schematic representation of a displacement of the surgical microscope of FIG. 1;

FIG. 3b shows a schematic representation of the dependence between a magnification and a detected displacement of the surgical microscope of FIG. 1 during a displacement according to FIG. 3a; and

FIG. 3c shows a schematic representation of a display of the surgical microscope of FIG. 1 after the displacement according to FIG. 3a.

In the exemplary embodiments described below, components that are alike in function and structure are indicated as far as possible by alike reference numerals. Therefore, to understand the features of the individual components of a specific embodiment, the descriptions of other embodiments and of the summary of the disclosure should be referred to.

### DETAILED DESCRIPTION OF EXEMPLARY EMBODIMENTS

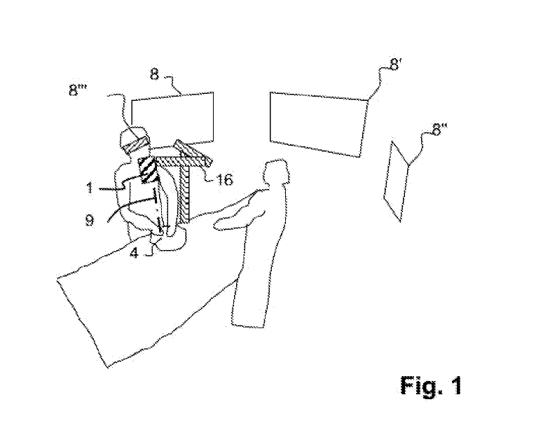

FIG. 1 shows a schematic representation of a digital surgical microscope 1 according to an embodiment of the invention used in the exemplary context of a surgical procedure.

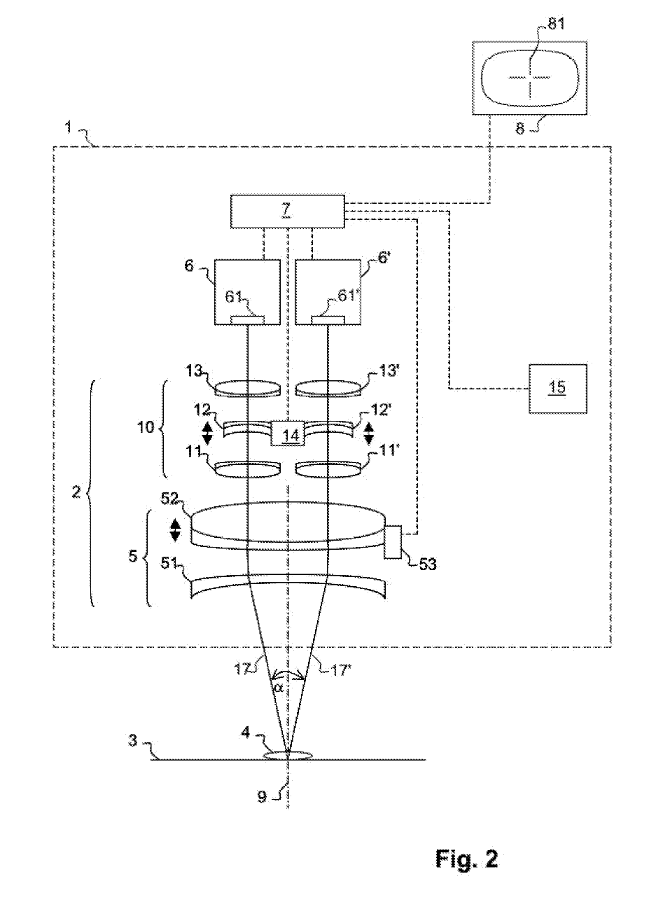

The surgical microscope 1 is supported by a floor stand 16 moveable on rollers (not shown), and by using the stand, a user can move the surgical microscope 1 manually such that an optical axis 9 of an objective lens (shown in FIG. 2) is directed onto an surgical area 4 to be imaged. The magnified image of the surgical area 4 generated by the surgical microscope 1 is output via lines (not shown) to three monitors 8, 8′, and 8″ and displayed on the monitors 8, 8′, and 8″. Furthermore, the magnified image of the surgical area 4 generated by the surgical microscope 1 is output via a radio interface to a head-mounted display 11′″ of a user.

As shown in the schematic representation of FIG. 2, the digital surgical microscope 1 of FIG. 1 is a stereoscopic microscope having an imaging system 2 that provides two optical imaging paths 17, 17′ intersecting in a focal plane 3 of the imaging system 2 of the surgical microscope 1 at a stereoscopic angle α. The size of the stereoscopic angle α depends on the respectively used working distance and amounts for the digital surgical microscope shown to between 6° and 10°.

In the embodiment shown, the imaging system 2 is comprised of a two-part objective 5 and a three-part zoom system 10. It is noted that the present invention is not limited to two-part objectives or three-part zoom systems, but may rather implement multi-part systems in general.

The two optical lenses 51 and 52 of the objective are consecutively traversed (passed through) by the two stereoscopic optical imaging paths 17, 17′. Each of the two optical lenses 51 and 52 of the objective is commonly traversed by the two stereoscopic optical imaging paths 17, 17′. The lens 51 located closest to the surgical area 4 to be imaged is a simple lens element while the other lens 52 is a doublet that can be moved between 200 mm and 450 mm relative to the lens 51 by a drive 53 for changing the working distance of the surgical microscope 1. A doublet comprises at least two optical lenses which are permanently bonded flat together, in particular by gluing, and which are made from materials with different refractive indices.

The three optical lenses 11, 12, 13 and 11′, 12′, and 13′ of the zoom system 10 are each doublets that are consecutively passed through by just one of the two stereoscopic optical image paths 17, 17′. The central lenses 12, 12′ of the zoom system 10 can be moved by a drive relative to the two outer lenses of the zoom system 10 for changing the magnification of the zoom system 10 between 8× and 20×.

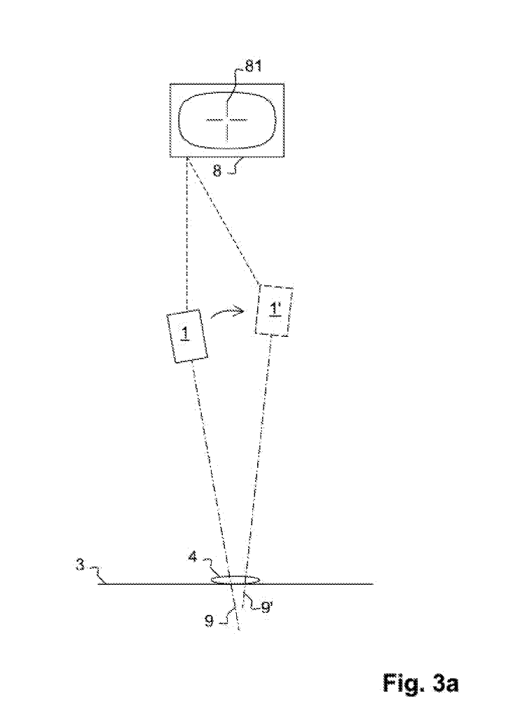

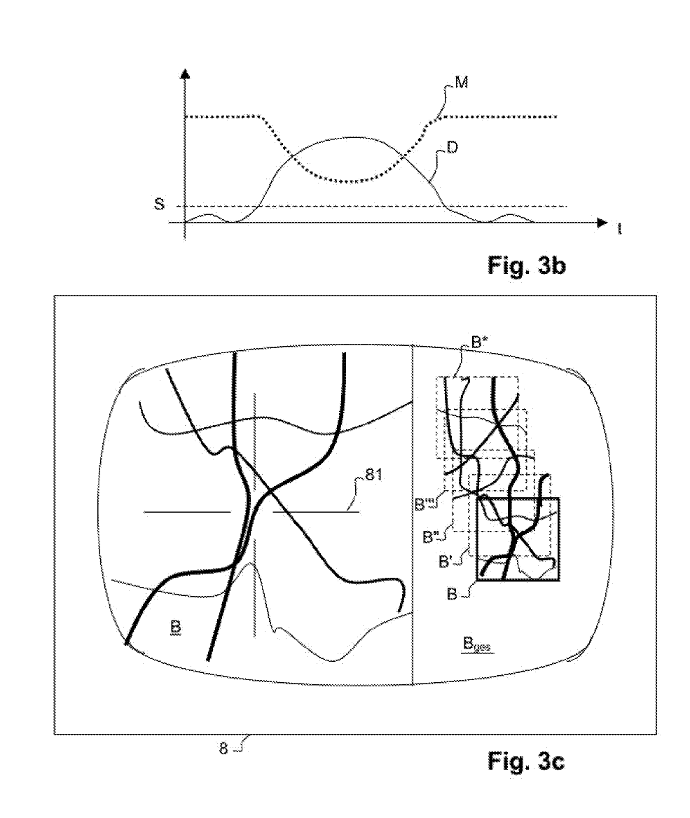

The imaging system 2 produces magnified images of the surgical area 4 along the optical image paths 17, 17′ on the reception areas 61, 61′ of two CCD sensors 6, 6′. The images of the surgical area 4 received on the reception areas 61, 61′ image the surgical area 4 at two slightly different angles. In the embodiment shown, the reception areas 61, 61′ each comprise a Bayer matrix providing a resolution of 1280×1024 image elements. Based on electrical signals output from the reception areas 61, 61′, the CCD-sensors 6, 6′ construct two-dimensional single images of the surgical area 4 imaged by the imaging system 2. The two-dimensional single images are received by the controller 7 and output to the at least one display 8. Although a total of four displays 8, 8′, 8″, and 8′″ is shown in FIG. 1, only one display 8 is shown in FIGS. 2 and 3 for the sake of clarity. Since the CCD-sensors 6, 6′ output two stereoscopic images, a 3D-monitor is actually used as display 8.

The controller 7, which is a processor configured by software, is in communication with the CCD-sensors 6, 6′, the drive 14 of the zoom system 10, the drive 53 of the objective 5, an acceleration sensor 15, and the at least one display 8 via data lines shown as dashed lines in FIG. 2 and FIG. 3a.

The controller 7 identifies identical image contents in single images following each other in time and being provided by the same CCD sensor and thus by the same optical imaging path, and compares the positions of the image contents with each other. A change in the position of the image contents is interpreted by the controller 7 as a displacement of the surgical microscope 1 transverse to the optical axis 9 of the objective lenses 51, 52 relative to the surgical area 4, and the distance D covered thereby in the focal plane 3 of the imaging system 2 is calculated based on the magnification used for the creation of the single images. The calculated distance D is compared to a threshold value of 0.25 cm specified by a user in the controller 7. This threshold value is valid for a distance of time between the single images of equal or less than one second.

Upon an exceeding of the threshold value, the controller 7 controls the drive 14 of the zoom system 10 such that the magnification M effected by the imaging system 2 as a whole decreases with increasing displacement speed of the surgical microscope 1 transverse to the optical axis 9 of the objective lenses 51, 52 relative to the surgical area 4 (and thus the distance of the displacement of the image contents in the single images calculated for a specified period of time), and vice versa. Upon falling short of the threshold, the controller 7 controls the drive 14 of the zoom system 10 such that the magnification M effected by the imaging system 2 as whole before the displacement is restored.

A displacement of the surgical microscope 1 relative to the surgical area 4 with a component of movement transverse to the optical axis 9 of the objective lenses 51, 52 is schematically shown in FIG. 3a. With the digital surgical microscope 1 shown, the decoupling of the image generation from the image presentation evidently enables the at least one display 8 to be stationary during the displacement.

The relation between the speed of displacement (the distance D covered within a respective period of time specified) and the magnification M is schematically illustrated in FIG. 3b. Line S indicates the threshold.

Controller 7 further stores the digital single images output from CCD sensors 6, 6′ automatically in a memory incorporated into the controller. Upon the threshold being exceeded, the controller 7 composes the stored single images to a total image of the imaged surgical area, and outputs the total image together with the current single image to the at least one display 8.

FIG. 3c schematically illustrates the display 8 after a displacement of the surgical microscope 1. The presently current image B is shown in the left field of display 8. Crosshairs 81 are superimposed to the current image by controller 7 during a displacement exceeding the threshold, with the crosshairs being positioned in the current image B presented on the display 8 such that the point in the surgical area 4 is marked, at which the optical axis 9 of the objective lenses 51, 52 intersects the surgical area 4 imaged. The total image B_{ges} constructed from the single images B*, B′″, B″, B′, B generated during the displacement that has exceeded the threshold is shown in the right field of the display 8. A frame highlights the current image B in the total image B_{ges}. The borderlines of the other single images B*, B′″, B″, B′ used for constructing the total image B_{ges} are illustrated in FIG. 3c with dashed lines, since they are not expressly shown on display 8.

The acceleration sensor 15 enables to distinguish between displacements caused by a displacement of an surgical area imaged and displacements caused by a displacement of the surgical microscope 1. A user may thus specify that certain displacements are not to be taken into account. In addition, this enables a determination of the surgical microscope 1 without requiring any image recognition. Accordingly, also a threshold for the acceleration is stored in controller 7.

Controller 7 controls drive 53 of the objective 5 continuously such that the surgical area imaged is always located in the focal plane 3 of the imaging system 2, and such that the imaging system 2 always provides a sharp image of the surgical area 4 imaged. By automatic image reversal and/or rotation of the single images, the controller 7 further ensures that the surgical area 4 imaged is presented on the at least one display 8 in the correct position.

While the disclosure has been described with respect to certain exemplary embodiments thereof, it is evident that many alternatives, modifications and variations will be apparent to those skilled in the art. Accordingly, the exemplary embodiments of the disclosure set forth herein are intended to be illustrative and not limiting in any way. Various changes may be made without departing from the spirit and scope of the present disclosure as defined in the following claims.

1. A surgical microscope, comprising:
an imaging system producing a magnified multidimensional image of an object disposed in a focal plane of the imaging system, the imaging system comprising an objective having at least one objective lens;
at least one image sensor disposed in an image plane of the imaging system and outputting an electrical signal representing the image of the object produced by the imaging system;
a controller receiving the signals output from the at least one image sensor, and converting the signals into digital single images of the object; and
at least one display in communication with the controller;
wherein the controller is configured to output the digital single images to the at least one display, to detect a displacement of the surgical microscope transverse to the optical axis (9) of the at least one objective lens relative to the object imaged, and to compare the displacement with a threshold; and
wherein the controller is further configured to perform at least one of the following steps provided the displacement detected exceeds the threshold:
control the at least one zoom system of the imaging system such that a magnification of the imaging system depends inversely monotonically from a relative velocity between the surgical microscope and the imaged object in a direction transverse to the optical axis of the at least one objective lens; and/or
store the digital single images, compose a total image of the object imaged from the single images stored, and output the total image to the at least one display,
provided the displacement detected exceeds the threshold.

2. The surgical microscope according to claim 1, wherein the controller is further adapted to detect at least one of the relative displacement and optional a speed of the surgical microscope transverse to the optical axis of the at least one objective lens by comparing temporally consecutive digital single images of the object produced from electrical signals of the at least one image sensor.

3. The surgical microscope according to claim 1, further comprising at least one acceleration sensor detecting at least one of the a relative displacement and optional a speed of the surgical microscope transverse to the optical axis of the at least one objective lens.

4. The surgical microscope according to claim 1, wherein the controller is further configured to control the at least one zoom system of the imaging system during the relative displacement of the surgical microscope transverse to the optical axis of the at least one objective lens such that the magnification of the imaging system is either inversely proportional to a relative velocity between the surgical microscope and the object imaged, or minimal.

5. The surgical microscope according to claim 1, wherein the controller is further configured to control the at least one zoom system of the imaging system after the relative displacement of the surgical microscope transverse to the optical axis of the at least one objective lens such that the magnification of the imaging system selected at the start of the displacement is restored.

6. The surgical microscope according to claim 1, wherein the controller is further adapted to control the objective of the imaging system during the relative displacement of the surgical microscope transverse to the optical axis of the at least one objective lens such that the object imaged is continuously positioned in the focal plane of the imaging system.

7. The surgical microscope according to claim 1, wherein the controller is further adapted to control the display during the relative displacement of the surgical microscope transverse to the optical axis of the at least one objective lens such that a marker is superimposed onto a center of the image presented on the display.

8. The surgical microscope according to claim 1, wherein the controller is further configured to control the display after the relative displacement of the surgical microscope transverse to the optical axis of the at least one objective lens such that the total image produced during the preceding displacement is presented in addition to the current single image produced from the signals output from the at least one image sensor.

9. The surgical microscope according to claim 1, wherein the controller is further configured to effect at least one of an image reversal and/or rotation on the single images produced from the signals output from the at least one image sensor.

10. The surgical microscope according to claim 1,
wherein the threshold is a minimum distance of the displacement of the surgical microscope relative to the object imaged of in particular at least 0.5 cm and further in particular of at least 1 cm.

11. The surgical microscope according to claim 1,
wherein the threshold is a minimum time period of the displacement of the surgical microscope relative to the object imaged of in particular at least 0.25 seconds, and further in particular of at least 0.5 seconds, and further in particular of at least 1 second.

12. The surgical microscope according to claim 4, wherein the controller is further configured to control the at least one zoom system of the imaging system after the relative displacement of the surgical microscope transverse to the optical axis of the at least one objective lens such that the magnification of the imaging system selected at the start of the displacement is restored.

13. The surgical microscope according to claim 4, wherein the controller is further adapted to control the objective of the imaging system during the relative displacement of the surgical microscope transverse to the optical axis of the at least one objective lens such that the object imaged is continuously positioned in the focal plane of the imaging system.

14. The surgical microscope according to claim 4, wherein the controller is further adapted to control the display during the relative displacement of the surgical microscope transverse to the optical axis of the at least one objective lens such that a marker is superimposed onto a center of the image presented on the display.

15. The surgical microscope according to claim 4, wherein the controller is further configured to control the display after the relative displacement of the surgical microscope transverse to the optical axis of the at least one objective lens such that the total image produced during the preceding displacement is presented in addition to the current single image produced from the signals output from the at least one image sensor.

16. The surgical microscope according to claim 4, wherein the threshold is a minimum distance of the displacement of the surgical microscope relative to the object imaged of in particular at least 0.5 cm and further in particular of at least 1 cm.

17. The surgical microscope according to claim 4, wherein the threshold is a minimum time period of the displacement of the surgical microscope relative to the object imaged of in particular at least 0.25 seconds, and further in particular of at least 0.5 seconds, and further in particular of at least 1 second.

18. The surgical microscope according to claim 6, wherein the controller is further adapted to control the display during the relative displacement of the surgical microscope transverse to the optical axis of the at least one objective lens such that a marker is superimposed onto a center of the image presented on the display.

19. The surgical microscope according to claim 6, wherein the controller is further configured to control the display after the relative displacement of the surgical microscope transverse to the optical axis of the at least one objective lens such that the total image produced during the preceding displacement is presented in addition to the current single image produced from the signals output from the at least one image sensor.

20. The surgical microscope according to claim 6, wherein the threshold is a minimum distance of the displacement of the surgical microscope relative to the object imaged of in particular at least 0.5 cm and further in particular of at least 1 cm.

21. The surgical microscope according to claim 6, wherein the threshold is a minimum time period of the displacement of the surgical microscope relative to the object imaged of in particular at least 0.25 seconds, and further in particular of at least 0.5 seconds, and further in particular of at least 1 second.

22. The surgical microscope according to claim 10, wherein the threshold is a minimum time period of the displacement of the surgical microscope relative to the object imaged of in particular at least 0.25 seconds, and further in particular of at least 0.5 seconds, and further in particular of at least 1 second.
